(19) United States
(12) Patent Application Publication
(10) Pub. No.: US 2004/0214234 A1
(43) Pub. Date: Oct. 28, 2004

(54) Method

(76) Inventors: Diana Clarke, Cambridge, MA (US); Jonas Frisen, Stockholm (SE)

Correspondence Address: JONES DAY, 222 EAST 41ST ST, NEW YORK, NY, 10017, US

(21) Appl. No.: 10/297,106

(22) PCT Filed: May 31, 2001
(86) PCT No.: PCT/GB01/02419

(30) Foreign Application Priority Data
May 31, 2000 (GB) 0013260.5

(51) Int. Cl. G01N 33/53; G01N 33/567; C12N 5/08

(52) U.S. Cl. 435/7.2; 435/366

(57) ABSTRACT

The present invention relates to a method to determine the differentiation potential of a target cell, said method comprising exposing the target cell ex utero to an early embryonic environment and detecting the progeny of said target cell. The invention further relates to the use of these methods to identify or provide cells for therapy or prophylaxis and the use of said cells in the manufacture of a medicamnent for use in therapy prophylaxis.

The present invention relates to a assay for testing the differentiation potential of a cell. In particular, the assay of the invention tests the full differentiation potential of a cell.

Stem cells having the capacity to differentiate into a variety of different cell types, have been the subject of much interest in recent years. Multicellular organisms are formed from a single totipotent (or pluripotent) stem cell. AS this cell and its progeny undergo cell divisions, the potential of the cells becomes restricted and they specialise to generate cells of a certain lineage. In several tissues a stem cell population is maintained in the adult organ, and it may generate new cells continuously or in response to injury. This feature has attracted interest, since it opens up the possibility of using stem cells to generate cell populations to use for therapy, for example to repair damage caused to tissues by disease or injury. This is of particular interest in the context of the nervous system.

Until recently, a “static” view prevailed on the fate of nerve cells in the central nervous system (CNS), that new neurons could not be generated in the adult mammalian brain. However, it has now been demonstrated by various groups that renewal of neurons and generation of the other cell types of neural tissues, astrocytes and oligodendrocytes, can take place in certain regions of the adult CNS (see e.g. R. McKay, Science 276, 6671 (1997); S. Weiss, et al., J. Neurobiol. 36, 307 (1998); S. Momma, et al., Curr. Opin. Neurobiol. 10, 45 (2000); L. S. Shiabuddin, et al., Mol. Med. Today 5, 474 (1999); S. Temple, et al., Curr. Opin. Neurobiol. 9, 135 (1999). This has opened up a quest for the neural stem cell, and a variety of reports have been published in the literature in this regard, including for example our earlier patent application WO 99/67363.

In particular, two stem cells have recently been identified in the adult central nervous system: ependymal cells (C. B. Johansson, et al., Cell 96, 703 (1999) and subventricular zone astrocytes (F. Doetsch, et al., Science 283, 534 (1999)), although it is not yet clear whether these two cell types represent independent populations or whether they share a lineage relationship (S. Momma, supra; B. A. Barres, Cell 97, 667 (1999)). These cells can be cultured as clonal cell aggregates referred to as neurospheres (B. A. Reynolds, et al., Science 255, 1707 (1992)).

Traditionally, the differentiation potential of stem cells in tissues of the adult has been thought to be limited to cell lineages present in the organ from which they were derived, but there is some evidence that some stem cells may have a broader differentiation repertoire. For example, neural stem cells isolated from the adult forebrain were recently shown to be capable of repopulating the hematopoietic system and producing blood cells in irradiated adult mice (C. R. Bjornson, et al., Science 283, 534 (1999)).

Others have reported myocyte generation in the embryonic mouse neural tube, as well as from a medulloblastoma cell line (S. Tajbakhsh, et al., Neuron 3, 813 (1994); N. L. Valtz, et al., New Biol. 3, 364 (1991)). In addition, there have recently been indications that other stem cell populations may not be restricted to generate cell types specific for the tissue in which they reside. Thus, marrow stromal cells transplanted to the brain can generate astrocytes (G. C. Kopen, et al., Proc. Natl. Acad. Sci. USA 96, 10711 (1999)). Moreover, hematopoietic stem cells can generate myocytes, and muscle progenitor cells can generate blood cells (E. Gussoni, et al., Nature 401, 390 (1999); K. A. Jackson, et al., Proc. Natl. Acad. Sci. USA 96, 14482 (1999)).

The potential to generate a variety of cell types from a given stem cell is potentially of very significant clinical importance, in that it may provide a ready means of generating a desired cell type for transplantation to treat different diseases and injuries. Accordingly, there is a need to be able to assay or assess the potential of a cell to differentiate into diverse different lineages, and in particular to assess its capacity to differentiate into lineages distinct from the tissue, from which the cell was derived. The present invention addresses this need.

Most of the available data indicate that progeny produced by nervous system stem cells are limited to neural cell fates (see e.g. R. McKay, supra; S. Weiss, et al., supra; S. Momma, et al., supra; L. S. Shiabuddin, et al., supra; S. Temple, et al., supra). For example, S. J. Morrison, et al., Cell 96, 737 (1999); P. M. White, et al., Development 126, 4351 (1999) have reported the lineage restriction of rodent neural crest cells.

However, the assay methods employed previously in the prior art such as discussed above were not designed to test the full developmental potential of the cells. Indeed, we believe that such assay methods of the prior art have not allowed for the full developmental potential of a cell to be expressed, and hence tested. In particular, we believe that the test conditions of such prior art systems have been such as to restrict the repertoire of progeny cells which might develop.

To address such problems, and to be able to determine or assess fully the developmental potential of a given cell, a novel assay has been developed according to the present invention, which is designed specifically to allow the full differentiation capacity of a given cell to be expressed and tested. This has been achieved by exposing the test cell to conditions which are conducive to, or which allow, the full differentiation potential of the test cell to be expressed or manifested.

In one aspect, the present invention provides a method to determine the differentiation potential of a target cell, said method comprising exposing the target cell ex utero to an early embryonic environment and detecting the progeny of said target cell.

In particular the present invention provides a method to determine the differentiation potential of a target non-ES cell, said method comprising exposing the target non-ES cell ex utero to an early embryonic environment consisting of embryoid bodies, or embryos or embryo cultures that have not completed gastrulation, for sufficient time to detect undifferentiated and differentiated progeny of said target non-ES cell.

Advantageously, the method of the invention permits the full or broad differentiation of a target cell to be determined.

The term “determine” as used herein includes all forms of assay or assessment of the differentiation potential of the target cell.

“Differentiation potential” is defined herein to include the differentiation capacity of a target cell, namely the potential of a cell to develop (or differentiate) into different cell types. This potential (i.e. potential capacity) to develop into one or more particular cell types may depend on the presence of signals, for example signalling molecules such as growth factors or other molecules (see further below), or indeed on the presence of a particular cellular environment. The cell types into which a given target cell may develop, may themselves be capable of further differentiation, or they may represent the end of a “differentiation lineage”. Thus, the differentiation potential is the developmental or differentiation capacity, or the plasticity of a cell, being the capacity to change (i.e. develop or differentiate) into a different cell type, which may be more organised or developed cell type, or a different type of cell altogether. Thus, the terms “differentiation potential” or “differentiation” as used herein also include the capacity of a target cell to become less specialised (or de-differentiated, or undifferentiated, or less differentiated) after which such cells may then develop into more specialised cells of the same or different cell type to the original target cell.

The “full” differentiation potential of a cell is the broadest possible differentiation potential of a target cell. In other words, it represents the full or maximum developmental repertoire or developmental capacity of a cell. Thus, one purpose or aim of the assay method of the invention is to test all, substantially all, nearly all, or all relevant of the different possibilities or potentials that a given target cell may have, including the capacity to differentiate into a cell of a different type, from that from which it is derived.

Thus, in one aspect, the present invention thus provides a method to determine the differentiation potential of a target cell, said method comprising exposing the target cell ex utero to an early embryonic environment which provides one or more or multiple signals enabling possible differentiation potentials of the target cell to be expressed, and detecting the progeny of said target cell. Ideally said methods will enable all possible differentiation potentials of the target cell to be expressed.

The “broad” differentiation potential of a cell includes the potential of a cell to differentiate into one or more cell types (e.g. two or more) which are distinct from the type into which the cell would, in its normal native environment, develop. In particular, the broad differentiation potential of a cell includes its differentiation potential when exposed to multiple (e.g. more than one) different signalling environments. This differentiation includes the differentiation to both extraembryonic and embryonic tissues (germ layers).

The assay method of the invention provides an environment for the target cell which provides signals for differentiation to diverse, and indeed, advantageously, to all possible lineages, including lineages distinct from which the target cell derives or originates. The environment of the assay method of the invention provides the appropriate and enabling conditions for full or broad differentiation potential to be expressed.

The “target cell” is the cell of interest or test cell. It may be any cell, including stem cells or more committed progenitor cells, embryonal carcinoma cells, transformed cells or cell lines, non-transformed continuous cell lines, or indeed a fully differentiated cell. A stem cell may be a multipotent, pluripotent or totipotent stem cell, able to differentiate into the full repertoire of progeny cells which are capable of being obtained within the organism from which the cell derives or originates. Alternatively, the cell may be a partly differentiated progenitor cell, already committed or dedicated to a particular lineage or lineages, but still capable of further differentiation to one or more cell types. The target cell may also be a cell regarded as fully differentiated, for example representing an “end point” in a particular lineage. By way of example, ependymal cells in the brain have previously been thought of as fully differentiated. However, recent work has shown that in fact ependymal cells may be stem cells in the adult CNS.

The target cell may be derived or obtained at any developmental stage from the source organism, for example it may be foetal, neo-natal, juvenile, adolescent or adult. It may also be derived from an artificial system such as a cell culture or cell line. The target cell may be native, or modified, for example genetically modified. The target cell may be obtained from any tissue or body part or body fluid of choice. Thus, the target cell may be of any dermal origin. The target cell may be any human or non-human animal cell, of any desired genus or species (e.g. any species in the animal kingdom, e.g. mammalian, avian, fish, reptile, insect etc.). In preferred embodiments of the invention the target cells are not embryonic stem cells (ES cells), i.e. are non-ES cells. In further preferred embodiments the target cells comprise stem cells derived from a post-natal or post-birth subject, a juvenile, adolescent or adult.

The target cell may be provided in any convenient way, for example as one or more isolated cells, which may be freshly isolated, or propagated, cultured or passaged etc. The target cell may be provided within or as part of a tissue, or fluid, or cell suspension, or may be contained in any desired or appropriate medium. Thus, the target cell may be provided as a target cell population in any desired or convenient way, according to techniques well known in the art. For example, target cells may be derived from initial cell isolates, primary cultures, amplified cultures of initial isolates or from clonal cultures. Neural stem cells may be provided as isolated cells or as neurospheres.

“Ex utero” means that the method excludes any steps involving implantation and development of an embryo in the uterus. In other words, the assay method of the invention does not, at any stage, involve any steps which are carried out in the uterus of a test animal. Thus, the target cell is not introduced into a uterus in any way, and in particular the early embryonic environment of the assay test system is not provided within, or transferred to, a uterus.

The term “early embryonic environment” as used herein defines an environment which provides or mimics the conditions of early embryogenesis. “Early embryogenesis” is defined as the period from the start of development of the embryo from the point of fertilisation to the completion of gastrulation. Functionally, the importance of this feature is that the early embryonic environment provides a signalling environment (or rather a multiplicity of signalling environments), which enables the full or broad differentiation potential of a cell exposed to that environment to be expressed. Thus, an environment of early embryogenesis advantageously provides the full range of possible signalling environments, which may direct cellular differentiation. In this state of early embryogenesis, the target cell is exposed to a large range and variety of possible differentiation-directing signals, for example inductive signals. An “inductive signal” is a signalling process or a signalling environment that acts directly or indirectly on a cell or group of cells to form a specific cell lineage or cell type. In particular, the target cell is exposed to a large variety of signalling environments which the cell is not normally, in its native state, exposed to. A signalling environment may be provided by molecular signals (e.g. molecules such as growth factors) expressed in the early embryogenic environment and/or by the cellular environment of the early embryonic environment, e.g. the cells which are present.

In particular, certain types of signalling occur during development, and are believed to contribute to a signalling environment according .to the present invention. Thus, a signalling environment may comprise two different types of signal or fate-controlling cue, firstly an extrinsic cue or signal, and secondly an intrinsic cue or signal.

The environment into which the cell is born (or finds itself in) and gradually differentiates, provides extrinsic cues (signals) which may comprise diffusible molecules, cell-membrane-attached factors, and extracellular-matrix bound signal molecules. These extrinsic cues generally activate signal transduction cascades and control differential gene expression in the recipient cell. However, the extrinsic cues are not always activators of signalling, and may also be so-called “transforming signals” i.e. inductive signals involving molecules that repress signals of primary activator signalling molecules. The ratio of activating and transforming signals is thought to be important in some types of cellular differentiation.

The cell itself, before the onset of differentiation, expresses or inherits from its precursor intrinsic cues (signals). Such intrinsic cues may include transcriptional activators or repressors. Intrinsic cues may be maintained by stem cell (or, according to the present invention, target cell) populations allowing them to respond when presented to a different/unique environment.

Cell fate is not a single. event decision, but consists of a multitude of potentially independent events that may be determined at different times and by different cues. In the assay system of the present invention, it is believed (although we do not wish to be bound by theory) that the signals the target cells receive from the early embryonic environment are inductive extrinsic signals (cues) that set up intrinsic signalling events in these cells, that may allow the cells to demonstrate a broader differentiation potential than would occur in their normal environment.

In the early embryonic environment, embryonic stem cells, in particular pluripotential embryonic stem cells, may be present.

As will be discussed in more detail below, the early embryonic environment may be provided by an embryo, or by an artificial system, e.g. an in vitro system such as an embryo culture or a preparation of embryoid bodies. Such an early embryonic environment may also be provided by embryonic stem cells. Thus in vivo, ex vivo and in vitro environments are encompassed. Preferably the early embryonic environment comprises or consists of embryoid bodies, embryonic stem cells, or embryos or embryo cultures that have not completed gastrulation. Preferably the target cells are exposed to said early embryonic environment for sufficient time to detect undifferentiated and differentiated progeny of said target cells.

In early development or embryogenesis of certain species, notably avian species, a “primitive streak” forms in the embryo. The “primitive node” is a small thickening at the cranial .end of the primitive streak. The primitive streak and primitive node may be considered as an elongated blastophore through which mesodermal material migrates inward. Later, the primitive node recedes caudally, inducing the organisation of mesoderm into notochord, somites, and lateral plates. The retreating primitive node is an organisation center that induces the reorganisation of the primitive streak material into the axial organs of the embryo. If the primitive node is transplanted to another location in the embryonic disc, a second complete body axis, including notochord, neural tube, and somites, develops at this site. The primitive node, therefore, corresponds to the notochord-mesoderm complex in the amphibian embryo in its function as organiser of the body axis. The primitive streak may thus be regarded as a general inducer, and the primitive node as a general organiser. Such inducers and organisers (including their developmental counterparts in other species) may provide or produce signals which contribute to a signalling environment according to the present invention. In the early embryonic environment of the present invention, therefore, inducers (e.g. primitive streak) and organisers (e.g. primitive node) may also be present.

Thus, according to the present invention, by its nature the early embryonic environment provides multiple signals for differentiation. In other words, two or more signals, or signalling environments are provided.

The nature of the early embryonic environment is such that all different germ layers (or dermal layers), both primitive and definitive, that contribute to the embryo proper and extraembryonic membranes, i.e. ectoderm, endoderm, and mesoderm, are developing, or have the potential to develop, and hence the environment has the capacity to develop or differentiate a full repertoire of cells of an organism, and is capable of providing a multiplicity (i.e. two or more) of signals for differentiation to diverse lineages.

As mentioned above, according to the present invention, the early embryonic environment may be provided a number of different ways, and a number of different embodiments of the method are possible.

In one such embodiment, the early embryonic environment may be provided by embryoid bodies. Embryonic Stem (ES) cells are totipotent and can be induced to differentiate to a variety of different cell types when cultured as embryoid bodies (G. M. Keller, Curr. Opin. Cell Biol. 7, 862 (1995)). The rationale behind this embodiment, is that such a culture of embryoid bodies would provide signals for differentiation to diverse lineages, and in the method of the invention, the capacity of such signals from ES cells to guide or influence differentiation of a target cell is evaluated. In this embodiment, the target cell is contacted with embryoid bodies. In particular, the target cell is co-cultured with embryoid bodies.

Thus, in this embodiment, the present invention provides a method for the determination of the differentiation potential of a target cell, said method comprising culturing said target cell with embryoid bodies, and detecting the progeny of said target cell.

Methods for the preparation and culture of embryoid bodies are well known in the art and described in the literature, (see for example, Cell Biology, a Laboratory handbook, Volume 1, Academic Press, edited by Julio E. Celis, 1994, Sanchez et al., in J. Biol. Chem., 260(33), 22419-22426, (1991) and Metzger et al., Cir. Res., 76, 710-719, 1995). ES or other cells for the formation of embryoid bodies may be obtained from any desired or convenient source. Thus, the species of the ES or other cells may be according to choice and may include for example embryonic stem cells or primordial germ cells from any metazoan species (e.g. invertebrates: annelids, arthropods, crustaceans, molluscs, echinoderms; vertebrates: fishes, amphibians, reptiles, birds, mammals; other: cnidarians; and poriferans). In the Example below mouse E14 ES cells were used to produce embryoid bodies.

The embryoid bodies may be derived from the same species or genus as the target cell under evaluation or from a different species or genus.

In general, embryonic stem (ES) cells (or generally any kind of stem cell or primordial germ cell, or any cell derived from these) are grown under culture conditions that will allow them to maintain the ability to form embryoid bodies (EBs). Such conditions are routine in the art. As EBs the cluster of cells have and are able to further initiate differentiation that results in the production of primitive ectoderm, endoderm, and mesoderm. The presence of these different germ layers allows the EB to produce signals to their own cells and also to other cells that may be in close contact with them including, according to the present invention, a target cell.

To form EBs, a representative method is discussed below. However, it will be understood by the skilled reader that modifications to such a method are possible. The basic steps of the method are to form embryoid bodies in a suitable culture system. The embryoid bodies are then collected and contacted with the target cell, e.g. a population of target cells. The target cells can be brought into contact with the embryoid bodies at any time after the embryoid bodies have formed, providing that the embryoid bodies retain their ability to provide the required early embryonic environment. Thus, the target cells may be brought into contact with the embryoid bodies immediately after their formation or the embryoid bodies may be kept in culture for a time period before the target cellsare brought into contact with them.

The ratio of target cell to embryoid body is not critical and may depend upon the particular embryoid body system and particular target cell used. For example, a range of 10:1 to 1:1 embryoid body to target cell (or target cell cluster), e.g. 6:1 to 2:1 e.g. 3:1 may be used. However, it may be necessary to modify such a ratio if, for example, the target cell secretes a signalling molecule that is inhibitory to the embryoid bodies' inducing activity at a given concentration. Such an effect can be compensated for by adding more embryoid bodies. Such modifications and determinations of ratios may readily be accomplished by the skilled worker in this field. The mixture of target cells and embryoid bodies is then cultured, according to standard culture techniques well known in the art. After culture for a suitable length of time (e.g. 1 minute to 100 hours, or more particularly 5 to 36 hours, e.g. 10 to 24 hours, e.g. 12 hours, or a sufficient amount of time to allow progeny cells to have developed, which can then be detected), the cell cultures may then be examined, to detect the progeny of the target cell. Again, time of culture is variable and may depend upon the particular system and/or target cell used. The detection step may be accomplished using standard and known techniques, as will be described further below. To facilitate the detection step, and depending on the culture conditions used, the culture of embryoid bodies and target cells may be plated, and allowed to grow as an adherent culture. For the detection step, the embryoid bodies may or may not be removed, depending on how the detection step is carried out (see further below).

The ES, stem or primordial germ cells i.e. the cells to be used to generate EBs, (along with the feeder layer of cells (if necessary) to maintain their potential) are trypsinized, rinsed in fresh culture medium (that maintains their totipotency) and cultured, for example placed on tissue culture grade plastic until the feeder cells have attached. The unattached ES cells (which do not attach quickly) are then removed and transferred to bacterial plates (non-tissue culture grade plastic that does not promote adhesion of cells) at clonal density, clustering density or plated as hanging drops of approximately 10 cells per drop on bacterial plates. All three of these conditions allow the formation of clusters of cells that adhere to their progeny and to each other (and not to the plastic). The cells are incubated for 12 hours under the above conditions in the same medium described above. The cells are then transferred to fresh medium identical to that described above with the exception that the medium does not contain factors that prevent differentiation to EBs (usually by removing Leukemia Inhibitor Factor (LIF)). The cell clusters are again plated on bacterial plates and maintained as non-adherent clusters of cells in the differentiation-promoting medium. The culture medium is changed every 24 hours. After approximately 8 days, the ES cells have formed clusters and cystic cylinders of cells roughly termed EBs based on the expression of primitive endoderm on their exterior surface (which can be visualised with any antibody that will recognise primitive endoderm). These cells are then collected and mixed homogeneously together with the target cell population being tested and allowed (approximately 3 EB:1 target cell (or target cell cluster) to settle randomly, but densely, by gravity into the bottom of a conical 1.5 ml tube in the medium specific for the target cell population being tested (preferably, a medium that will also allow the survival of the EBs for at least 12 hours). These densely packed clusters of cells are placed upright in the conical tube in a normal humidified tissue culture incubator at 37° C./5% CO₂ for twelve hours. After twelve hours, the cells are plated onto tissue culture grade plastic or onto a suitable substrate or an adherence substrate that promotes specific differentiation. This could include, for example, poly-ornithine, fibronectin, laminin, gelatin, or any combination of substrates. For detection, and if one wishes to remove the EB cells, twelve hours after plating, selection against the EB cells is begun with an appropriate antibiotic or selection factor that will only allow elimination of the EBs from the cultures. However, removal of the EBs is not an absolute necessity as long as one could conclusively identify the target cell population being evaluated (e.g: human vs. mouse cells). At this time specific differentiation factors may also be added to the target cell medium to allow survival and differentiation of the target cells and/or their progeny. The cultures are subsequently maintained in the target cell medium containing the factors and the medium may be changed every 48 hours to 50% fresh medium: 50% conditioned medium. The cultures can be evaluated to detect or test for target cell progeny (e.g. evaluated for specific differentiation markers, see further below) as long as the cultures can be maintained in vitro.

Example 1 below includes a representative example of the “embryoid body” embodiment of the method of the present invention. As is described further below, results obtained using this assay system show that adult neural stem cell-derived cells can adopt a muscle fate in vitro.

In an alternative embodiment of the assay method of the invention, the early embryonic environment may be provided by an avian or amphibian embryo. Any avian or amphibian species may be used, but fowl represent convenient sources and chick embryos may conveniently be used, as they are easily available.

Using birds or amphibians has the advantage that eggs can conveniently be used to provide embryos in a situation where they develop outside the mother, and hence where they can be readily, conveniently and speedily grown in an in vivo-like situation, and where access to the embryo is readily available and simple, thereby permitting easy manipulation of the embryo.

Thus, an alternative embodiment of the method of the invention provides a method for the determination of the differentiation potential of a target cell, said method comprising introducing said target cell into an avian or amphibian embryo at any time up to the completion of gastrulation of said embryo, allowing said embryo to develop and detecting the progeny of said target cell.

As mentioned above, the avian or amphibian embryo is conveniently contained in an egg, which is laid, and therefore outside the mother's body, and the embryo may conveniently be allowed to develop, simply by incubating the egg.

It is an important feature of the invention that the target cell is introduced prior to or up to the completion of gastrulation, e.g. at any time during the gastrulation process. This is significant, because a target cell added at this time is able to join in the gastrulation process and is thereby exposed to various signalling environments in the different germ layers of the embryo (ectoderm, endoderm, mesoderm).

Advantageously, the target cell is introduced into the amniotic cavity of the embryo. This allows target cells to integrate into the primitive ectoderm that faces the amniotic cavity and to become distributed in the definitive ectoderm, endoderm and mesoderm during gastrulation. The target cell is thus exposed to various signalling environments in the different germ layers.

At the time an egg is laid, an embryonic disc has formed comprising approximately 60,000 cells. The cells begin to organise in preparation to initiate gastrulation as soon as an egg is placed at an incubating temperature. The incubation temperature is ideally 39.5° C., but other temperatures may be used, e.g. in the range of 11° C. to 43° C., for example a temperature of about 37° C. may be used. A lower temperature (e.g. lower than 39.5° C.) may for example be used if it is desired to increase the exposure to inductive signals, by slowing development rate etc.

The egg can be utilised in the method of the invention from prior to incubation, through to the end of gastrulation. Thus, the target cell can be introduced to the embryo at any time, beginning from introducing the cell to the unincubated embryonic disc present at the time the egg is laid, and at any time to the gastrulating embryo (epiblast) at any time during the gastrulation process until gastrulation is complete. In an avian embryo, for example, the target cell may be introduced on the involuting primitive streak or on the embryonic disc (epiblast) at the site the primitive streak will form when gastrulation begins.

Thus, the target cell may be introduced to the avian or amphibian embryo prior to or during the initiation of gastrulation or even during the late stages of gastrulation.

Introduction of the target cell may take place in any convenient way, according to techniques well known in the art. Thus, the target cell may be added to the embryo, for example by placing the cells on the embryo. The target cell may be introduced onto or into the primitive streak or into or onto the gastrulating epiblast or into or onto the forming mesodermal or endodermal layers. The target cell may be introduced by injection, for example using a micropipette, or any delivery system that can be used to place cells in or on the above-mentioned regions of the embryo.

The introduction of the target cell may take place at any time point from freshly laid egg (prior to incubation) or from stage 1 of the embryo up to stage 12, more particularly up to stage 6, for example at or up to stage 4. See Hamburger and Hamilton, “A series of normal stages in the development of the chick embryo”, p. 49-92, for a description of stages of the chick embryo.

Following introduction of the target cell, the embryo is allowed to develop. As mentioned above, this is conveniently achieved by incubating the egg. The embryo may be allowed to develop to a time point prior to or after hatching or to a metamorphic phase such as a tadpole or even into fully differentiated adulthood. The egg may be incubated at incubating temperature (e.g. at 11° C. to 43° C. as described above).

Example 1 below describes an assay of this embodiment of the invention utilising chick embryos. Results show that neurospheres derived from single cell cultures of adult neural epithelial cells have the broad potential to generate progeny of various lineages.

In a still further embodiment of the invention, the early embryonic environment may be provided by an embryo culture. In this embodiment embryos may be cultured in vitro according to techniques known in the art (see for example Hsu in Developmental Biology, 76, 465-474, 1980 and “Guide to techniques in mouse development”, edited by Wassurman and DePamphilis, Methods in Enzymology, Vol. 325, 1993, and Manipulating the Mouse Embryo, a laboratory manual, 2nd Edition 1994, Cold Spring Harbor Laboratory Press, edited by Hogan et al.).

Accordingly, this aspect of the invention provides a method for the determination of the developmental potential of a target cell, said method comprising introducing said target cell into a cultured embryo and detecting the progeny of said target cell.

The cultured embryo may be cultured in vitro from after fertilisation to up to and including the in utero equivalent of 11 dpc (days post conception). this time period includes preblastulation up to early organogenesis. For example, culture may be up to the in utero equivalent of 4, 5, 6, 7, 8, 9 or 10 dpc.

In advantageous embodiments, the target cell may be introduced at any time up to completion of gastrulation of the embryo.

In this embodiment of the assay of the invention, embryos may be isolated from the uterus of the donor animal (e.g. mouse). The embryo is dissected free from the decidua, extraembryonic membranes, and Reichert's membrane. The embryo is then placed in a suitable medium under appropriate conditions, depending on the nature of the embryo, e.g. in a chemically defined medium at 37° C. and at oxygen levels which are changed in accordance to the metabolic requirement of the embryos at different ages. Development beyond the primitive streak stage of gastrulation requires use of a combination of roller and rotating drum methods with daily changes of fresh medium containing freshly prepared human umbilical cord serum.

In a mammalian (e.g. mouse) embryo culture system, the target cell may be introduced onto or into any appropriate part or surface of the embryo. In particular, the target cell may be introduced onto or into the embryoblast of the blastocyst, the bilaminar embryonic disc of the implanting embryo, or into the amniotic cavity or onto the primitive streak up until the end of gastrulation.

The target cell may be introduced by any convenient means, as for the avian/amphibian assay system described above, for example by injection e.g. with a micropipette.

Following introduction of the target cell, the embryo culture may be continued up to the equivalent of 11 dpc (early organogenesis or the “hind limb bud”stage, also referred to as the 29-31 somite stage), or up to any of the dpc stages mentioned above.

As for the other embodiments, the progeny of the target cell may then be detected in the cultured embryo. (see further below).

The embryo for culture may be of any desired or convenient genus or species, for example mammalian embryos, e.g. rodent (e.g. mouse or rat) embryos may be used as well as embryos from any metazoan species. The embryo may be of the same or different species or genus as the target cell.

In order to determine the differentiation potential of the target cell, the progeny of the target cell are detected.

In embodiments of the invention where embryos or embryo cultures are used to provide the early embryonic environment the localization or location of the progeny cells in the embryo or embryo culture may also be determined.

The term “progeny” as used herein refers to any cell which is derived or differentiated or developed from a target cell. Said progeny cells may be derived from the target cells as a result of the first or any subsequent cell divisions which the target cell undergoes, or may in fact comprise the target cells themselves which have undergone differentiation or development without cell division. As discussed above, the target cells may become more or less specialised when exposed to an early embryonic environment in the methods of the invention described herein. Thus, the progeny of the target cells may be undifferentiated, de-differentiated or differentiated compared to the target cells, or in other words may be more or less differentiated than the target cells. The progeny cells may themselves be capable of further differentiation or may represent the end of a differentiation lineage. The progeny cells may comprise cells which are more or less organised cell types of the same type as the target cells or may comprise different cell types altogether from the target cells. For example the target cell type may be derived from the adult nervous system, for example may be an adult neural stem cell, and the progeny cells may comprise one or multiple cell types with one or more characteristics of for example muscle cells, kidney cells, heart cells, various cell types of the nervous system and/or epithelial cells of various types.

The term “detected” is used broadly herein to include all methods of detecting the presence of, or analysing, or identifying the progeny of the target cell.

Differentiation potential may be assessed or determined in many different ways. Thus, for example, the ability of the target cell to contribute to the formation of different tissues may be detected by following the fate of the progeny cells. In other words, in the context of an assay which uses an embryo, the progeny may be detected by examining different tissues of the embryo, to see in which tissues progeny of the target cell reside. Thus, in certain embodiments, detection of the target cell progeny may take place by detecting characteristics of the target cell. For example, if the target cell is of a particular species (e.g. human or mouse), or of different allotypes within a species (e.g. inbred mouse strains) different to that of the embryonic host environment, it may be detected by detecting particular species- or allotype-specific markers e.g. murine or human markers (e.g. the mouse-specific epitope H-2 K^{b}). Alternatively, the target cell may be provided with an artificial marker system, for example the target cell may be obtained from a transgenic animal incorporating a marker or reporter gene (i.e. a genetic marker) which encodes a detectable or signal-giving product. For example, in the Examples below ROSA26 mice are used (G. Friedrich, et al., Genes Dev. 5, 1513 (1991)), the target cells of which express β-galactosidase, enabling identification of their progeny by X-gal histochemistry or with antibodies. Alternatively, the target cells (and hence any progeny derived therefrom) may express a gene which enables them to be selectively isolated, e.g. from a co-culture or embryo, for further study. Such genes may include those encoding resistance e.g. to an antibiotic, which would enable cells not expressing such a marker to be selectively eliminated, leaving viable the progeny of the target cell. For example, ROSA26 cells also express a neomycin resistance gene.

Thus, one way of detecting target cell progeny is to detect a marker of the target cell. This may be detected in different tissues of an embryo (in vivo or in embryo culture), thereby yielding information as to which tissues the target cell progeny have contributed to. Alternatively, once target cell progeny have been detected, they may further be examined for markers of differentiation. Thus, cells of particular types or in particular tissues, frequently carry markers (e.g. express molecules e.g. cell surface or intracellular proteins, or carbohydrate-structures etc.) characteristic of that cell type or tissue. For example, myocytes express desmin, mesonephric tubule cells express Pax2, endodermal cells express an endoderm specific epitope, TROMA-1, cytokeratin 20 is expressed in intestinal epithelium, and albumin in liver. Other examples of differentiation markers include glial cell fibrillar associated protein (GFAP) for astrocytes, Beta III Tubulin for neurons, keratin for epithelial cells, alpha feto protein for liver, and myosin heavy chain for cardiac and skeletal muscle. Such markers may also be morphological, (for example muscle cells are striated). Thus, in this way, the progeny of the target cell may be examined or analysed to determine, to what tissue or cell types they have differentiated, or indeed, whether or not they have differentiated to a particular cell or tissue type.

Such molecular or morphological markers are well known in the art and widely described in the literature, and new markers are continuously documented in the literature. Techniques for detecting and identifying such markers are also well known and widely available, and may be based for example on histochemistry, e.g. immuno-histochemistry, or other immunological systems, and molecular biology techniques such as in situ hybridisation or RT-PCR.

Thus, in detecting the target cell progeny, it can be detected whether or not the target cell has differentiated to particular cell or tissue types, and whether it has differentiated into a cell type different from itself. Conveniently, it can be tested or detected whether or not the target cell has differentiated into 2 or more, or 3 or more, different cell types. It may be detected whether or not the target cell has differentiated into a cell of a different dermal origin from itself, and it may further be detected whether or not progeny cells of all three different germ or dermal layers (endoderm, mesoderm, ectoderm) have developed.

It may also be determined whether the target cell can also differentiate to cell types of the original origin of the target cell, for example in the case of brain stem cells, whether they may become or contribute to new but different classes of neurons than those they would have developed into normally. It is also possible that neural stem cells may develop or differentiate into a different classification of the nervous system (e.g. CNS to PNS and/or ANS).

The invention as described herein is also directed to methods wherein target cells and/or progeny cells of the target cells are isolated. Additionally progeny cells obtainable, preferably obtained, by the methods of the invention described herein form further aspects of the invention.

The term “isolated” as used herein in connection with the isolation of target cells and/or progeny cells refers to the separation or removal or isolation of said cells from other cells which may be present in the co-culture or embryo. Preferably all or substantially all the desired target cells and/or progeny cells will be isolated from the undesired remaining cells. However, biological systems are by their nature variable, and 100% separation/isolation may not always be achieved and indeed is not always necessary. However, preferably at least 30%, 40%, 50%, 60%, 70%, 80%, 90%, 95% or 99% of said target cells and/or progeny cells are isolated.

The isolation may be carried out by any suitable method. For example the separation may be carried out by physical means, for example by using affinity separation techniques (for example magnetic bead sorting) to separate the target and/or progeny cells from the remaining cells on the basis of cell surface markers which are expressed on the target and/or progeny cells but not on the undesired cells. Alternatively the cells to be isolated may be labelled with visual markers which can be detected directly or indirectly, for example fluorescent markers and the isolation carried out on the basis of these markers, using for example FACS analysis. A convenient way of fluorescently labelling cells is to engineer the cells to express a fluorescent protein such as green fluorescent protein (GFP). Indeed engineering the cells to express any gene that encodes for a protein that can be used to enable sorting of the cells using e.g. flow cytometry or magnetic bead sorting can be used in the isolation of cells.

Appropriate methods of isolation are not limited to the use of markers found on the target and/or progeny cells but can equally be carried out using natural or induced markers expressed on the cells it is desired to remove, i.e the cells providing the early embryonic environment, e.g. the non target derived cells making up the embryoid body, embryos or embryo cultures. Such cells may be transfected in vitro to express or may be otherwise engineered to contain a transgene expressing a suitable marker so that the “negative” cells (in this case the desired target and target derived cells) can be kept after sorting and further propagated. In addition, the cells providing the early embryonic environment can be transfected in vitro to express or may be otherwise engineered or derived from a transgenic animal to contain a transgene expressing a suicide gene (e.g. a thymidine kinase (TK) gene) so that after treatment with an appropriate agent (gangcyclovir in the case of a TK gene) the undesired cells would be killed leaving only the cells originating from the target cell population. Also the target cells (and their progeny) can be selected by inducing the death of the non-desired cells. This could readily be carried out if the target cells and their progeny included resistance genes, for example antibiotic resistance genes as described above.

Once the required populations of target cells and/or progeny cells have been isolated these can then be used directly, for example used directly for therapy or other in vivo or in vitro methods. Alternatively these cells can be further processed or manipulated before they are subject to the desired use. Examples of further processing include further culture or further differentiation, or genetic manipulation or alteration of the cells to for example render the cells more useful or especially adapted for the desired application, for example the desired therapy. In particular, when the therapy involves the transplantation of cells into subjects, genetic alterations may be carried out in order to make the cells less susceptible of rejection.

As mentioned above, the methods of the invention have clinical utility in providing cells for therapy. Such methods may be used to generate a variety of useful cell types for example for the treatment of various diseases or injuries. In particular such methods may be used to generate one or more desired cell types for transplantation to treat different diseases and injuries.

In addition, the methods of the invention can be used for diagnostic purposes, e.g. for the diagnosis of whether or not certain target cells will be useful for certain therapies or for providing or generating cells for certain therapies. For example, certain patients or subjects may have stem cells (or other target cells) that are not appropriate or do not have the potential for use or to provide or generate cells for use in certain therapies, e.g. certain patients may have stem cells that are not capable of developing into the required cell type for therapy. Thus, a subjects stem cells (or other target cells) could be tested using the methods of the present invention (i.e. used as target cells in the methods of the present invention) in order to “diagnose” whether or not these cells have the potential to be used in a specific therapy (e.g. in autotransplantation). Such “diagnosis” would generally be carried out by detecting the location and/or the cell type of the progeny cells and determining whether appropriate types of progeny cell have been produced.

Thus, a yet further aspect of the present invention provides the methods of determining differentiation potential of target cells as defined herein, wherein said methods are used for diagnostic purposes, or are used to “diagnose” the potential of the target cells to be used in methods of therapy or prophylaxis as discussed herein.

Thus, yet further aspects of the invention provide the use of the methods of the invention to identify or provide or diagnose cells for therapy or prophylaxis or to identify or provide or diagnose cells for the generation of therapeutically- or prophylactically-useful cells. Preferably said therapy or prophylaxis involves transplantation of the cells identified, provided or generated. More particularly, the methods of the invention may be used to identify and determine appropriate cell types to be used for the generation of therapeutically- or prophylactically-useful cells. Once generated such cells, for example appropriate stem cells, can be administered directly to a patient and allowed to differentiate in vivo or can be differentiated in vitro, using for example the methods of the invention and then administered.

Thus, it can be seen that a yet further aspect of the invention provides cells identified, diagnosed, provided or generated by the methods described herein for use in therapy or prophylaxis. Use of such cells in the manufacture of a medicament for use in therapy or prophylaxis are also included.

Viewed alternatively the invention further provides a method of treatment or prevention of disease in a subject, comprising administering to said subject an appropriate amount of cells identified, diagnosed, provided or generated by the methods described herein.

Examples of diseases which may be treated or prevented in accordance with the present invention include any diseases which are susceptible to treatment by cellular therapy, e.g. any diseases which can be treated by organ or tissue transplantation, the treatments of which generally have associated therewith the problems of such conventional organ or tissue transplantation. Specific examples of such diseases include heart disease (e.g. ischemic injuries or other atrophies), skeletal muscle atrophies (e.g. Duchenne Muscular Dystrophy), liver disease (e.g. hepatitis, need for tissue replacement after resections due to cancer), diabetes, (preferably type I diabetes), etc. The methods of the invention are also useful in treating neurodegenerative diseases such as for example Parkinson's disease, Alzheimer's disease and Amyotrophic lateral sclerosis.

The cells when used in therapy or prophylaxis may be administered as a composition with one or more physiologically acceptable carriers or excipients. Generally such cells will be administered in a suspension containing a physiologically acceptable buffer or other liquid. The cellular compositions will generally be sterile and as free as possible from contaminating materials, such that they are suitable for administration to a subject. They will generally be administered to the appropriate body site by injection. Methods of formulating cells for administration and appropriate components of these formulations would be well known to a skilled person in the art and any appropriate method or components may be used. The concentration/dosage of cells required in any formulation will depend on the intended therapeutic or prophylactic application and other factors such as the weight of the patient and mode of administration. Appropriate doses would be readily determined by a person skilled in the art. The cells may be formulated and/or administered with other therapeutic agents which are effective in the treatment or prophylaxis of the particular disease concerned. Such agents may for example be an agent which influences the performance of the cells after they have been administered. For example agents which increase cell survival, reduce rejection of the cells by the immune system, or induce the migration, differentiation or proliferation of the cells in situ may be used. Specific examples include anti-apoptotic agents, cell specific growth factors, free radical scavengers and immunosuppressants. The cells may be co-administered with these other therapeutic agents or may be administered sequentially or separately, as appropriate.

The invention also has utility in research and study of developmental and differentiation processes. For example, the effects of genes and molecular substances, e.g. drugs, on development and differentiation may be studied.

In this regard, the methods of the invention can be used to develop assays for testing the effects or side effects on cells of various compounds or compositions which may be candidates for pharmaceutical development. In such embodiments a particular or selected target cell is taken through the methods of the invention, after which the progeny cells may optionally be isolated and the effect of one or more compounds on the progeny cells determined by exposing the cells to the compounds. Such methods could be used for example to test whether a candidate drug was likely to display undesired side effects to a particular progeny cell type, for example was toxic to the cell type, or whether a candidate drug displayed desired advantageous effects on the cell type.

Such methods thus provide a relevant screening test for compounds which is easy to perform and can be carried out in vitro, in vivo or ex vivo by selection of the appropriate experimental systems from for example the embryoid body, embryo or embryo culture systems described herein. Such methods may provide useful, relevant, quick and cost effective pre-clinical screening for candidate drugs.

In a more particular example of such methods, an adult human or mouse stem cell, for example a neural stem cell, might be used as a target cell and induced to differentiate using e.g. the chick embryo system described herein, into various progeny cell types, including for example heart, kidney and muscle progenitor cells. The cells deriving form the chick are then eliminated using isolation methods for example as described above and one or more of the progeny cell types can be used to assay test compounds or drugs. In this way for example drugs which have particular advantageous or disadvantageous effects on heart cells (or any other progeny cell type) could be screened.

Thus, a yet further aspect of the invention provides a method of assaying the effect of compounds or compositions on cells, said method comprising any of the methods to determine the differentiation potential of target cells or the methods of inducing target cells to differentiate as described herein and further comprising a step wherein the target cells and/or the progeny cells are exposed to said compound or composition and the effect thereof on the cells assessed.

From the above description and discussion it can be seen that the methods of the invention described herein can further be used to induce target cells to differentiate into different cell types. Preferred methods and conditions for inducing this differentiation are discussed above. Thus, a further aspect of the invention provides a method of inducing target cells to differentiate into different cell types, said method comprising exposing the target cell ex utero to an early embryonic environment.

Such differentiated cells can be subjected to any of the uses, for example use in therapy and prophylaxis described herein. Preferably and advantageously the differentiated cells are isolated from the remaining/unwanted cells. Methods for carrying out such isolation of cells are also described above. Additionally, differentiated cells obtainable or obtained by such methods of inducing target cells to differentiate are also included within the scope of the invention.

The invention will now be described in more detail in the Examples below, with reference to the drawings in which:

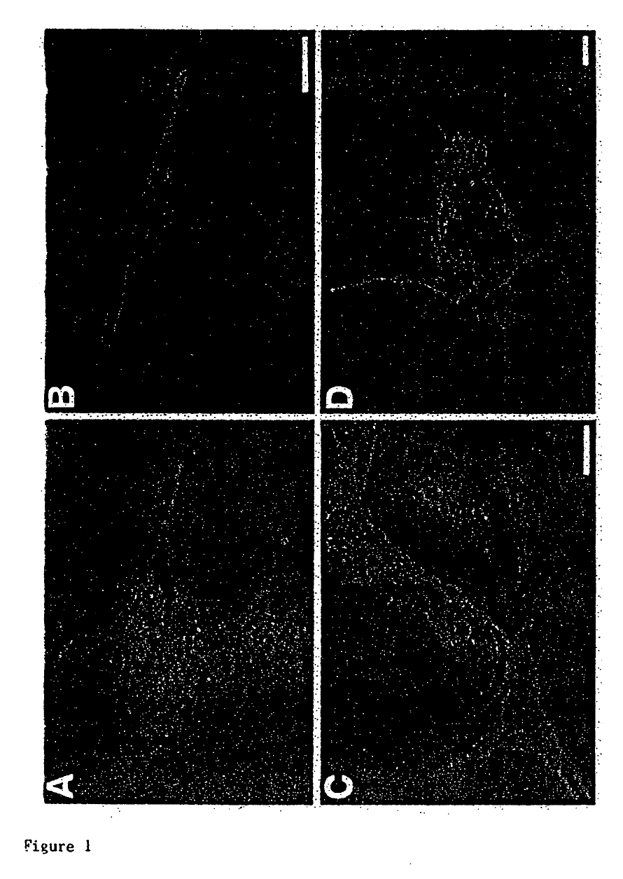

FIG. 1 shows the generation of myocytes from adult neural stem cells. Neural stem cell clones from ROSA26 mice were cultured with embryoid bodies to induce differentiation; the ES cells were subsequently eliminated by G418 selection. A subpopulation of β-galactosidase-immunoreactive (A) neural stem cell progeny are elongated and show immunoreactivity for the muscle cell marker desmin (B). Some neural stem cell derived cells are myosin heavy chain immunoreactive and show varying degrees of fusion and syncytia formation (C, D). Nuclei are visualized with blue Hoechst stain in (D). Scale bars are 20 μm in B and C and 50 μin D.

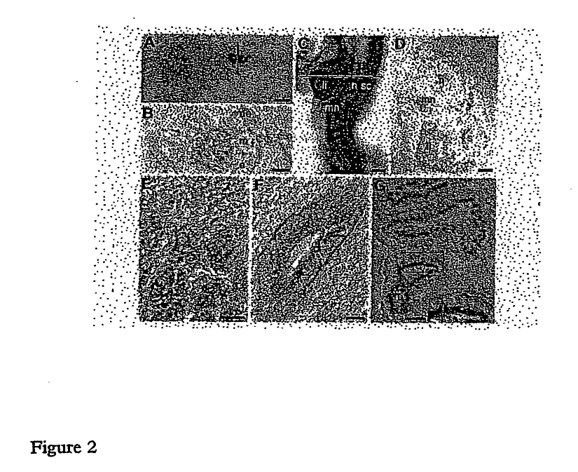

FIG. 2 shows that adult neural stem cells contribute to the formation of several organs in chick embryos. (A) shows whole mount X-gal staining of a stage 8 chick embryo, visualizing scattered blue neural stem cell derived cells. The line indicates the plane of section of the same embryo shown in (B). Neuroectoderm (ne), mesoderm (m) and endoderm (e) are indicated. (C) shows the trunk region of a cleared highly chimeric stage 23 embryo. A cross section of the embryo in (C) (plane of section indicated by line) is shown in (D). Liver (li), notochord (n), spinal cord (sc) and mesonephros (mn) are indicated. (E) Neural stem cell derived cells visualized with an antibody against the mouse specific epitope H-2 Kb intermingled with host cells in the liver of a stage 23 chick embryo. In (F) the epithelium of the stomach shows H-2 Kb-immunoreactivity (brown) and X-gal staining (blue). In (G), cytoplasmatic expression of lacz in mesonephric tubule cells (blue) show nuclear Pax2-immunoreactivity (red, higher magnification from area in box). Scale bars are 250 μm in A and D, 50 μm in B, 500 μm in C and 25 μm in E-G.

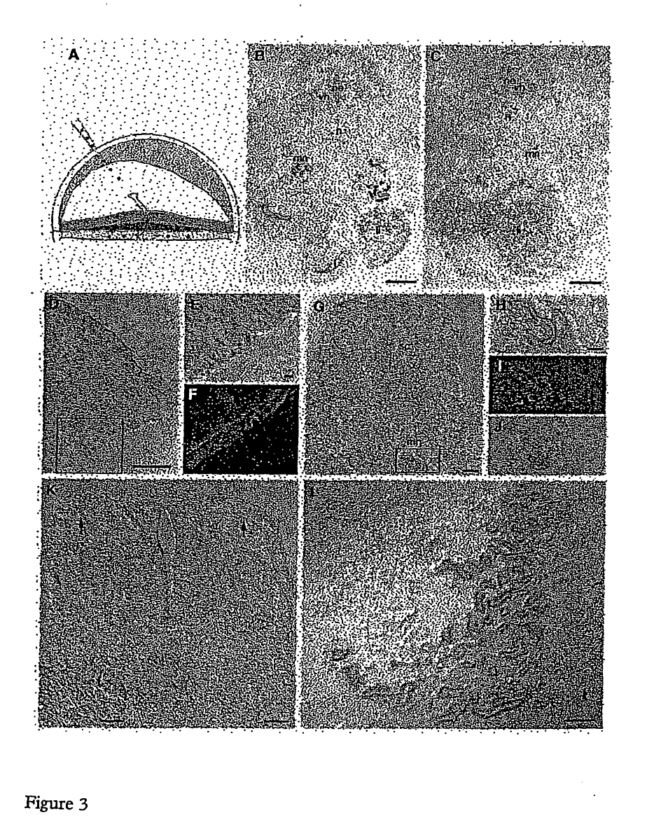

FIG. 3 is a schematic drawing showing the injection of ROSA26 adult neural stem cells into the amniotic cavity of the gastrulating chick embryo (A). (B) and (C) show examples of cross sections through the trunk region of two stage 23 chick embryos. Contribution of adult neural stem cell progeny (blue) is prominent in spinal cord neuroepithelium ine) and ventral horns (vh), mesonephros (mn), notochord (n), liver (li), stomach (s) and intestine (i). In (D) scattered lacZ expressing cells are detected in the epidermis of a stage 23 chick forelimb by X-gal staining. The area in the box is shown at higher magnification in (E), and keratin-immunoreactivity in that area is shown in (F). (G) Stage 23 chick embryo with a low degree of chimerism with lacZ expressing neural stem cell derived cells only in a mesonephric tubule on one side. In (H), a detail from (G) (indicated by box) is shown at higher magnification, and (I) and (J) show two adjacent sections labelled with antibodies against β-galactosidase and H-2 Kb, respectively. In (H-J), a subpopulation of cells in one mesonephric tubule are labelled, whereas an adjacent tubule is negative. (K) shows a cross section of a stage 23 chick embryo, and arrows point to lacZ expressing cells in the dermamyotome of the somites. The left somite is shown at higher magnification in the inset. In (L) numerous tubules containing lacZ expressing epithelial cells are intermingled with unlabelled tubules in a stage 36 chick embryo. Scale bars are 400 μm in B and C, 50 μm in D, 5 μm in E and F, 100 μm in G and L, 40 μm in H and inset in K and 200 μm in K.

### EXAMPLE 1

In this Example, the differentiation potential of neural stem cells from the adult mouse brain is studied using two different assay systems, firstly an assay based on culture with embryoid bodies, and secondly using chick embryos.

Methods

The anterior portion of the lateral wall of the lateral ventricles of adult mouse brain were dissected out and enzymatically dissociated in 0.7 mg/ml hyaluronic acid, 0.2 mg/ml kynurenic acid and 1.33 mg/ml trypsin in HBSS with 2 mM glucose at 37° C. for 30 minutes and then passed through a 70 μm nylon mesh (Falcon). The cells were centrifuged at 200 g for 5 min, resuspended in 0.9 M sucrose in 0.5× HBSS and centrifuged for 10 min at 750 g to remove myelin fragments. The cell pellet was resuspended in 2 ml of culture medium, placed on top of 10 ml 4% bovine serum albumin (BSA) in EBSS solution and centrifuged at 200 g for 7 min. The culture medium consisted of 20 ng/ml EGF (Collaborative Biomedical Products), B27 supplement (Life Technologies), 8 mM HEPES, 2 mM glutamine, 100 U/ml penicillin, and 100 μg/ml streptomycin in DMEM-F12 medium (Life Technologies). EGF (20 ng/ml) was added to the medium every 48 hours after the initial plating of the cells. Primary neurospheres were passaged to generate secondary neurospheres, which were used in all experiments, except where stated otherwise.

For clonal experiments, single cells were transferred with a micropipette to the microwells of 96-well plates and allowed to form small neurospheres. They were then dissociated into single cells which were again plated singly and allowed to grow to small neurospheres. Each neurosphere was then were dissociated separately into a dish and allowed to form small neurospheres again. Two such neurospheres were then injected into the amniotic cavity of stage 4 chick embryos. The wells were inspected after each transfer of the cells and occasionally wells containing more than one cell were omitted from the analysis. The single cells were cultured in 50% fresh culture medium and 50% neurosphere conditioned medium filtered through a 0.2 μm filter.

For selection of ependyma-derived neurospheres, mice were anaesthetized with chloral hydrate (400 mg/kg) and 3 μl 0.2% w/v DiI (1,1′-dioctadecyl-6,6′-di (4-sulfophenyl)-3,3,3 ′, 3′-tetramethylindocarbocyanine, Molecular Probes) in DMSO was injected stereotaxically into the right lateral ventricle. The injection coordinates were 0.5 mm posterior 0.3 mm lateral to Bregma and 2.2 mm below the dura mater. Cultures were established from the contralateral ventricle as described above 3 hours after the injection. ES cells (E14 clone) were cultured under standard conditions (A. L. Joyner, Ed., Gene targeting. A practical approach, Oxford University Press, Oxford, 1993) and embryoid body formation was induced by changing the medium to DMEM containing 20% FCS. Small secondary neurospheres were cultured for 12 hours together with simple embryoid bodies in neurosphere medium in eppendorf tubes to ensure close contact with the ES cells. The cells were then plated on gelatin or fibronectin coated tissue culture plates and after 12 hours the ES cell derived cells were eliminated from cultures by addition of 300 μg/ml G418. This resulted in complete elimination of the ES cells within 8 days. We observed that generation of non-neural cell types only occurred when the stem cells were added as small neurospheres to the ES cells, but not if the spheres were dissociated, and therefore used secondary neurospheres in all experiments.

For immunohistochemistry, cultured cells or cryostat sections were incubated with primary antibodies 1 hour at 37° C. or overnight at 4° C., rinsed in PBS, and incubated with secondary antiserum for 45 minutes at room temperature. The following primary antibodies were used: mouse monoclonal anti-muscle myosin heavy chain (Developmental Studies Hybridoma Bank, D. Bader, T. Masaki, D. A. Fischman, J. Cell Biol. 95, 763 (1982)), mouse monoclonal anti-H-2 Kb conjugated to biotin (Clone AF6-88.5, PharMingen), mouse monoclonal anti-desmin (Dako), rabbit anti-Pax2 (Berkeley Antibody Company), and goat anti-β-galactosidase (Biogenesis).

White Leghorn eggs were incubated to stage 4 and 10% india ink was injected under the blastoderm to visualize the embryo. Neurospheres (4-5) were injected into the amniotic cavity through drawn glass capillaries. Manipulated eggs were incubated for an additional 1-9 days before analysis. X-gal histochemistry was performed as described (A. L. Joyner, Ed., Gene targeting. A practical approach, Oxford University Press, Oxford, 1993).

Results and Discussion

To evaluate the capacity of the signals from ES cells to guide the differentiation of neural stem cells, we cultured adult neural stem cells together with embryoid bodies. The neural stem cells, derived from ROSA26 mice (G. Friedrich, et al., Genes Dev. 5, 1513 (1991)), express β-galactosidase enabling identification of their progeny by X-gal histochemistry or with antibodies against β-galactosidase (C. B. Johansson, et al., Cell 96, 25-34 (1999), and see also “Methods” above and FIG. 3). Moreover, ROSA26 cells express the neomycin resistance gene, which allowed us to later eliminate the G418 sensitive ES cells from the co-cultures and specifically study the remaining resistant neural stem cell derived cells. When cultured with embryoid bodies, neural stem cell progeny was frequently found to display immunoreactivity for desmin (FIGS. 1A, B), an intermediate filament protein expressed by myocytes (E. Lazarides, et al., Proc. Natl. Acad. Sci. USA 73, 4344 (1976)). Moreover, many of the neural stem cell derived cells fused to form muscle cell-like syncytia and showed immunoreactivity to the myocyte protein, myosin heavy chain (D. Bader, et al., Neuron 13, 813 (1994)) (FIGS. 1C, D). These cells did not display any signs of cellular transformation. Neural stem cells cultured under the same conditions, but in the absence of ES cells, never expressed these markers nor did they form syncytia. We did not find evidence for differentiation to endodermal cell fates by immunohistochemistry with an antibody against an endoderm specific epitope (TROMA-1, data not shown). This observation is consistent with the fact that endodermal differentiation is rare in embryoid body cultures, in contrast to muscle cell differentiation which is commonly observed. These data demonstrate that adult neural stem cell derived cells can adopt a muscle fate in vitro.

In order to analyze the differentiation potential of adult neural stem cells in vivo, we next assayed their ability to contribute to the formation of different tissues by introducing them into the early embryonic environment and following the fate of their progeny. We injected adult mouse brain neural stem cells into the amniotic cavity of stage 4 chick embryos (see “Methods” above). We reasoned that this may allow some neural stem cells to integrate into the primitive ectoderm that faces the amniotic cavity and to become distributed in the definitive ectoderm, endoderm and mesoderm during gastrulation (FIGS. 2A-B). The cells would then be exposed to various inductive environments in the different germ layers. Of the embryos that survived the injection of the neurospheres (26%), 24 out of 109 were chimeric and contained lacZ positive cells derived from the adult neural stem cells. No chimeric embryos were derived from injections of dissociated neurosphere cells. The specificity of the X-gal staining in chimeric embryos was supported by overlapping immunohistochemical labeling with antisera against β-galactosidase. The murine origin of these cells was further confirmed by overlapping immunoreactivity to the mouse specific epitope H-2 Kb (FIG. 2F). In addition to the nervous system, where reproducibly a high degree of chimerism was seen, lacZ expressing cells were frequently found in mesodermal derivatives such as the mesonephros and notochord, as well as in epithelial cells of the liver and intestine, which are of endodermal origin (Table 1, FIG. 2). In the tissues containing lacZ expressing cells, there was a mosaic pattern of labeling with a varying proportion of neural stem cell derived cells (FIG. 2). The lacZ expressing cells had indistinguishable morphology from surrounding host cells, and expressed markers normally present in cells of the particular tissue, for example Pax2.in mesonephric tubule cells (FIG. 2G) and keratin in epidermal cells. To test whether a single neural stem cell may have the potential to generate progeny that can differentiate into various cell types, we established clonal cultures by transferring single cells from dissociated neurospheres to microwells with a micropipette. Neurospheres derived from single cell cultures were tested in the chick assay and were found to have the same broad differentiation potential as described above, demonstrating that a single adult neural stem cell has the potential to generate progeny of various lineages. To specifically test the differentiation potential of an identified neural stem cell, we cultured cells from the lateral ventricular wall of animals that had received an injection of the fluorescent dye DiI. In such cultures, DiI labeled neurospheres from ependymal cells (C. B. Johansson, et al., Supra) were isolated with a micropipette and injected into the amniotic cavity of chick embryos. These ependyma-derived neural stem cells showed the same broad differentiation potential described above (data not shown).

Although we reproducibly found neural stem cell progeny in various organs in chick embryos, there were other tissues which never contained lacZ expressing cells. For example, we failed to detect contribution to the hematopoietic system. This is intriguing since the adult neural stem cells can differentiate along this lineage after transplantation to irradiated adult mice (C. R. Bjornson, et al., Science 283, 534 (1999)). It is well established from ES cell studies that the strain background of the ES and host cells have a large influence on the degree of chimerism in specific tissues and that certain strain combinations result in no or very low chimerism in certain organs (J. D. West, Curr. Top. Dev. Biol. 44, 21 (1999)). Thus, although the ES cells are totipotent, they fail to display their full potential in certain situations. In line with this, it appears that the neural stem cells may have a broader differentiation potential than revealed in the chick assays.

The data presented here suggest that stem cells in different adult tissues may be more similar than previously thought and perhaps in some cases have a developmental repertoire close to that of ES cells.

TABLE 1
Frequency of chimerism in different chick tissues
containing neural stem cell progeny

| Tissue | |
|---|---|
| Ectodermal | |
| Forebrain | 71% (17/24) |
| Midbrain | 71% (17/24) |
| Hindbrain | 71% (17/24) |
| Spinal cord | 96% (23/24) |
| Epidermis | 79% (19/24) |
| Mesodermal | |
| Notochord | 96% (23/24) |
| Mesonephric epithelium | 92% (22/24) |
| Mesonepbric mesenchyme | 92% (22/24) |
| Somites | 71% (17/24) |
| Heart muscle | 38% (9/24) |
| Endodermal | |
| Lung epithelium | 45% (9/20) |
| Stomach epithelium | 83% (20/24) |
| Stomach wall | 83% (20/24) |
| Intestinal epithelium | 96% (23/24) |
| Intestinal wall | 83% (20/24) |
| Liver | 92% (22/24) |

1. A method to determine the differentiation potential of a target cell, said method comprising exposing the target cell ex utero to an early embryonic environment and detecting the progeny of said target cell, wherein said target cell is introduced into the early embryonic environment which is provided by an amphibian or avian embryo, embryoid bodies, or embryonic stem cells, and further wherein said target cell is not an embryonic stem cell.

2. The method of claim 1, wherein said early embryonic environment comprises embryonic stem cells.

3. The method of claim 1 or claim 2 wherein the early embryonic environment is provided in vivo, ex vivo or in vitro.

4. The method of any one of claims 1 to 3 wherein the early embryonic environment consists of embryoid bodies, embryonic stem cells, or embryos that have not completed gastrulation.

5. The method of any one of claims 1 to 4 wherein the early embryonic environment provides one or more signals enabling possible differentiation potentials of the target cell to be expressed.

6. The method of any one of claims 1 to 5 wherein the target cell is derived from a neo-natal, juvenile, adolescent or adult source.

7. The method of any one of claims 1 to 6, said method comprising culturing said target cell with embryoid bodies, and detecting the progeny of said target cell.

8. The method of any one of claims 1 to 7 wherein the embryoid body is produced from embryonic stem cells.

9. The method of any one of claims 1 to 8 wherein the embryoid bodies are derived from the same or different species or genus as the target cell.

10. The method of any one of claims 1 to 6, said method comprising introducing said target cell into an avian or amphibian embryo at any time up to the completion of gastrulation of said embryo, allowing said embryo to develop and detecting the progeny of said target cell.

11. The method of claim 10 wherein the embryo is a chick embryo.

12. The method of any one of claim 10 or claim 11 wherein the embryo is contained in an egg and the embryo developed by incubating the egg.

13. The method of any one of claims 10 to 12 wherein the target cell is introduced into the amniotic cavity of the embryo.

14. The method of any one of claims 10 to 13 wherein the target cell is introduced onto or into the primitive streak or into or onto the gastrulating epiblast or into or onto the forming mesodermal or endodermal layers of the embryo.

15. The method of any one of the preceding claims wherein the localisation of said progeny of the target cell is determined in the embryo.

16. The method of any one of the preceding claims wherein detection is performed by detection of species-, allotype-, cell and/or differentiation specific markers.

17. The method of any one of the preceding claims wherein said target cells and/or said progeny are isolated.

18. A method as claimed in any one of claims 1 to 17 wherein said methods are used for diagnostic purposes.

19. A method as claimed in any one of the preceding claims to identify or provide or diagnose cells for therapy or prophylaxis or to identify or provide or diagnose cells for the generation of therapeutically- or prophylactically-useful cells.

20. A method as claimed in claim 19 wherein said therapy or prophylaxis involves transplantation of the cells identified, provided, diagnosed or generated.

21. A method as claimed in claim 19 or 20 to identify and determine appropriate cell types to be used for the generation of therapeutically- or prophylactically-useful cells.

22. Cells as defined in claim 19 for use in therapy or prophylaxis.

23. Use of cells as defined in claim 19 in the manufacture of a medicament for use in therapy or prophylaxis.

24. A method of treatment or prevention of a disease in a subject, comprising administering to said subject an appropriate amount of cells as defined in claim 19.

25. A method of inducing target cells to differentiate into different cell types, said method comprising exposing the target cell ex utero to an early embryonic environment, wherein said target cell is introduced into the early embryonic environment which is provided by an amphibian or avian embryo, embryoid bodies, or embryonic stem cells, and further wherein said target cell is not an embryonic stem cell.

26. The method as claimed in claim 25, wherein the method is as defined in any one of claims 1 to 18.

27. The method of claim 25 or 26, wherein the differentiated cells are isolated.

28. A method of assaying the effect of compounds or compositions on cells, said method comprising the methods as defined in any one of claims 1 to 18 or claims 25 to 27, and further comprising a step wherein said target cells and/or the progeny thereof are exposed to said compound or composition and the effect thereof on the cells assessed.
